(19) United States
(12) Patent Application Publication
(10) Pub. No.: US 2008/0139527 A1
(43) Pub. Date: Jun. 12, 2008

(54) METHODS FOR TREATMENT OF HEART DISEASE

(76) Inventor: Kota J. Reddy, Houston, TX (US)

Correspondence Address: PATTERSON & SHERIDAN, L.L.P., 3040 POST OAK BOULEVARD, SUITE 1500, HOUSTON, TX, 77056, US

(21) Appl. No.: 11/608,503

(22) Filed: Dec. 8, 2006

(51) Int. Cl. A61K 31/397; A61K 31/4406; C12Q 1/60; A61P 9/00

(52) U.S. Cl. 514/210.02; 435/11

(57) ABSTRACT

Methods relate to preventing, limiting and/or reversing coronary heart disease, such as heart disease caused by atherosclerosis. These methods for cardiac wellness include administration of therapeutically effective amounts of cholesterol altering medications to a patient in combination with a restricted diet intake of the patient such that the patient attains a high-density lipoprotein (HDL) cholesterol level higher than a low-density lipoprotein (LDL) cholesterol level. Further, this greater than one ratio of HDL to LDL due to the restricted diet intake and selection of sufficient dosages of the medications can indicate a reversal in heart disease of the patient.

### BACKGROUND OF THE INVENTION

1. Field of the Invention

Embodiments of the invention generally relate to treatment of heart disease.

2. Description of the Related Art

Blood cholesterol levels correlate to a risk of developing atherosclerosis and coronary heart disease, which create an increased danger of heart attacks and strokes. Blood transports cholesterol in a modified form of lipoproteins because of insolubility of the cholesterol. Two primary forms of the lipoproteins include low-density lipoprotein (LDL) and high-density lipoprotein (HDL). Excess amounts of the LDL in the blood leads to arteries becoming at least partially blocked by depositions forming cholesterol plaques along the inner wall of the artery. The plaque that causes the atherosclerosis inhibits blood flow due to accumulation and can create catastrophic events if the plaque ruptures.

In contrast to the LDL, which is known as “bad” cholesterol, higher levels of the HDL lower the risk of heart disease, thereby giving the HDL the name “good” cholesterol. The HDL helps with disposal of excess cholesterol by carrying to the liver the LDL from other tissues within the body. Based on the foregoing, general guidelines such as those provided by the National Cholesterol Education Program (NCEP) of the National Institutes of Health (NIH) set desirable, acceptable and undesirable standards for total cholesterol levels, HDL levels, a total cholesterol-to-HDL ratio, and LDL levels. For example, these guidelines set a desirable level of the HDL at more than 60 milligrams per deciliter (mg/dL) and concentrations optimal for the LDL at less than 100 mg/dL. A person having an HDL level lower than the LDL level fits into criteria defining current accepted optimal blood cholesterol concentrations. While present cholesterol lowering treatments can therefore aim to lower an LDL to HDL ratio, the HDL level remains lower than the LDL level with these treatments based on the accepted optimal blood cholesterol concentrations.

Along with low cholesterol consumption diets, various medications aid in altering the blood cholesterol levels. The medications control the blood cholesterol levels by regulating, for example, absorption of dietary supplied cholesterol, biosynthesis of cholesterol itself and its esterified forms, metabolic removal of circulating cholesterol, and excretion of cholesterol via bile and feces. Effects of the various medications can include lowering the LDL level and/or increasing the HDL level. Utilizing one or sometimes two of the cholesterol altering drugs along with a low cholesterol diet provides limited success rates in treating heart disease. Further, the treatments that alter the blood cholesterol levels can slow atherosclerosis progression, but lack any applications designed to prevent heart disease or enable active plaque regression with treatments to reverse atherosclerosis.

Therefore, there exists a need for improved methods of treating heart disease. A further need exists for a method of reversing heart disease and detecting the reversal.

### SUMMARY OF THE INVENTION

Embodiments of the invention generally relate to preventing, limiting and/or reversing coronary heart disease, such as heart disease caused by atherosclerosis. These methods for cardiac wellness include administration of therapeutically effective amounts of cholesterol altering medications to a patient in combination with a restricted diet intake of the patient such that the patient attains a high-density lipoprotein (HDL) cholesterol level higher than a low-density lipoprotein (LDL) cholesterol level. Further, this greater than one ratio of HDL to LDL due to the restricted diet intake and selection of sufficient dosages of the medications can indicate a reversal in heart disease of the patient. In some embodiments, addition of a lipid regulating medication, such as fish oil or a fibrate (e.g, fenofibrate and gemfibrozil) assists in transfer of the LDL from pattern B to the larger size of pattern A.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 shows pathways for combination therapies according to embodiments of the invention.

FIG. 2 illustrates an exemplary flow diagram of methods according to embodiments of the invention.

### DETAILED DESCRIPTION

Embodiments of the invention relate to treatments designed to prevent and/or reverse heart disease in a patient. New uses of compositions of matter, which define cholesterol altering drugs or medications, aid in controlling levels of high-density lipoprotein (HDL) and low-density lipoprotein (LDL) according to aspects of the invention as described herein. The treatments utilize a combination of medications and diet in order to enable the patient to attain a greater amount of the HDL than the LDL. In other words, the patient obtains an HDL:LDL ratio greater than one.

For some embodiments, effective dosages of the medications described hereinafter provide sufficient amounts to maintain the LDL level at less than about 60 milligrams per deciliter (mg/dL), preferably less than about 50 mg/dL, and more preferably less than about 40 mg/dL. Maintaining the HDL level at more than about 60 mg/dL can ensure that the HDL:LDL ratio remains greater than one. Other goals for some embodiments include the patient attaining one or more of the following: i) at least 35% of the HDL being HDL2b; ii) substantially all of the LDL with a particle configuration of LDL pattern A; iii) apolipoprotein B (ApoB) concentration less than about 60 mg/dL; and iv) apolipoprotein A1 (ApoA1) at an amount more than about 150 mg/dL. Such results can be obtained by the patient within a short period of time, often in three to six months. Following the treatment for an amount of time, preferably at least six months and more preferably at least a year, can lead to a significant reversal of plaque with the help of accelerated reverse cholesterol transport. As further shown herein through data obtained regarding patients, the patients treated according to embodiments of the invention suffered no cardiac events, such as heart attacks.

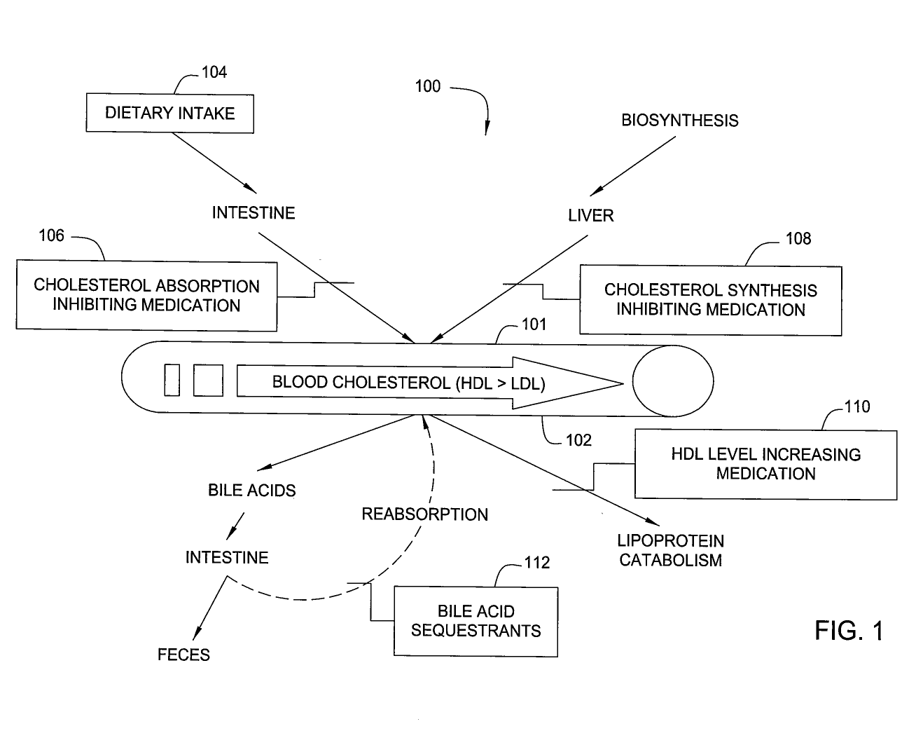

FIG. 1 schematically shows pathways 100 for combination therapies according to embodiments of the invention in order to achieve the aforementioned goals regarding blood cholesterol (illustrated by arrow 101) flowing through a circulatory vessel 102 of the patient. Treatments include restricting a dietary intake (i.e., any and all solid/fluids consumed) 104 of the patient as described in further detail hereafter. According to some embodiments, treatments utilize at least three drugs each selected from different ones of four major categories of medications including cholesterol absorption inhibitors 106, liver cholesterol synthesis inhibitors 108, HDL level increasers 110, and bile acid sequestrants 112. Low doses of each of the drugs relative to effective monotherapy dosages of just one of the drugs enable the medications taken in combination to achieve desired results thereby limiting potential detrimental side effects.

A first cholesterol synthesis inhibiting medication such as 3-hydroxy-3-methylglutaryl CoA (HMG-CoA) reductase inhibitor (e.g., a statin such as atorvastatin, cerivastatin, fluvastatin, itavastatin, lovastatin, pravastatin, rosuvastatin, rivastatin, and simvastatin) decreases the production of cholesterol in the liver. A second cholesterol absorption inhibiting medication such as azetidinones (e.g., ezetimibe), beta-sitosterol, stanol esters, and sterol glycosides (e.g., tiqueside and pamaqueside) prevents absorption of cholesterol in the intestine. A third HDL level increasing medication such as nicotinic acid or salts and derivatives thereof raises HDL levels and can also change cholesterol particle sizes from small to large and reduce lipoprotein(a) (Lp(a)). Fibrates such as gemfibrizol and fenofibrate can also aid in increasing the HDL level and assisting in transfer of small particle to large particle size of the LDL. Optionally, a fourth bile acid sequestrant medication such as cholestyramine, colesevelam, colestipol, colestilan, and dialkylaminoalkyl derivatives of a cross-linked dextran blocks reabsorption of bile from the intestines during digestion when taken with food.

First, the diet for the patient substantially reduces the intake of simple carbohydrates, primarily simple sugars and starches. Acceptable foods for consumption include complex carbohydrates (e.g., vegetables, beans, lentils, legumes and whole grains) and monounsaturated/poly-unsaturated fats (e.g., olive oil, canola oil, flaxseed oil, grapeseed oil, walnuts, hazel nuts, almonds, cashews, Brazil nuts, avocados, sesame seeds, fish oil and pumpkin seeds). Further, the diet limits intake of saturated fats to less than 10 grams a day and avoids trans fats. Examples of foods containing saturated fats include: animal fats (bacon, sausage, bologna, pastrami, salami, dark meat poultry, poultry skin, hog dogs (beef or pork), pork ribs, ground pork, pork sausage fatty beef), plus coconut oil, palm oil, palm kernel oil, cottonseed oil, regular dairy products (butter, cream, half-and-half, cream cheese, sour cream, cheese, ice cream, whole milk), lard, and shortening.

Vegetables, beans, legumes and/or lentils make up about 50% of the solids that the patient consumes. The remaining half of the solids can come from fat free or lean meats, spices, brown rice, wild rice, brown basmati rice, egg whites, egg substitute, rolled oats, regular oats, steel-cut oats, tofu, soy products, nuts, avocados, olives, low sugar soymilk, no sugar yogurt, flax seed oil, fish oil and/or 70% sugar free saturated fat free dark chocolate powder. Regarding liquids, the diet permits drinking water (preferable), tea, coffee, and/or up to two diet sodas a day.

The criteria for the diet prevents dietary intake of certain foods. For some diets, the patient cannot consume dairy products unless no fat and no sugar, juices, fruits, regular sodas, fish, breads, pastas, potatoes, white rice, alcohol and fried foods. Additionally, the diet sets total calories of the acceptable foods for dietary intake proportionate with height, sex and exercise of the patient.

Benefits of applying the diet include weight loss and reduction in triglycerides, blood sugars, and hemoglobin A1c. When these changes happen, the HDL cholesterol level goes up, LDL particle size converts from small to large particle LDL, and percentage of HDL2b (large HDL particles) goes up. With respect to LDL particle size, ApoB represents a component of cholesterol indicative of the size of the LDL particles since this component corresponds to small LDL traits. Since small particle size LDL tends to deposit along artery walls, reducing ApoB levels correlates to improvements in heart disease risk. By contrast, large LDL traits due to their size and buoyancy within blood flow tend not to contribute as much to atherosclerosis. Regarding detection of ApoA1 and HDL particle size, ApoA1 converts to HDL3 (small particle size), which then either converts to HDL2b or small particle size LDL. However, the diet tends to block conversion of the HDL3 to the LDL since such metabolic reactions require presence of sugars and starches. Therefore, high levels of ApoA1 subsequently supply the HDL3 that the diet causes to be preferentially transformed into the HDL2b with its larger size providing a larger surface area to more readily pickup LDL within the arteries.

Medications along with the diet lower the LDL level of the patient to below an identified maximum value, such as less than about 50 mg/dL. Once the LDL level is less than about 50 mg/dL, the process of atherosclerosis slows down as evidenced by animals other than humans almost never developing heart disease and having an LDL level of less than about 60 mg/dL. Furthermore, the likelihood of plaque formation in humans drops to almost zero if the LDL level is less than 40 mg/dL. Therefore, the identified maximum value for the LDL level aids in slowing or stopping additional plaque buildup.

Administering both a cholesterol synthesis inhibiting medication, such as a statin, and a cholesterol absorption inhibiting medication, such as ezetimibe, to the patient helps achieve the lowering of the LDL level. The statin decreases production of cholesterol and increases available LDL receptors to help lower amounts of the LDL that is circulating. Normal metabolic processes increase available LDL receptors to compensate for the reduced synthesis of cholesterol. When the liver is prevented from making cholesterol as occurs with use of the statin, a majority of patients become hyper absorbers and increase absorption of cholesterol from the gut. However, ezetimibe use blocks absorption of cholesterol from the gut including any possible increases in absorption that may secondarily result from the statin use.

Blocking cholesterol reabsorption helps achieve cholesterol excretion and lowers the LDL level to below the identified maximum value. Every day the liver releases bile acids made from the LDL in the blood, but substantially all of the bile acids are reabsorbed after traveling to the intestine where the bile acids are used to digest certain foods. Administering a bile acid sequestrant, such as colesevelam hydrochloride, aids in excretion of the bile acids and hence the cholesterol consumed in synthesis of the bile acids. The excretion of the cholesterol forces the liver to attempt to generate more cholesterol in order to make more bile acids. However, the cholesterol synthesis inhibiting medication makes generating more cholesterol impossible. Next, the body tries to increase absorption of cholesterol, which process is blocked by the cholesterol absorption inhibiting medication. Consequently, the expression of LDL receptors increases due to normal metabolic compensating processes, thereby increasing absorption of the LDL circulating in the blood to assist in making more bile acids. The result of the bile acid sequestrant therefore aids in dropping the LDL to below the identified maximum value.

In some embodiments, addition of a fifth further lipid regulating medication, such as fish oil or a fibrate (e.g, fenofibrate and gemfibrozil), to a combination of a statin, ezetimibe, colesevelam hydrochloride, and nicotinic acid assists in transfer of the LDL from pattern B to the larger size of pattern A. Patients with elevated small dense LDL levels after initial treatment with statin, ezetimibe, colesevelam hydrochloride, and nicotinic acid can thus benefit from administering of the fish oil or the fibrate. The fibrate also lowers ApoB levels, reduces triglycerides and increases HDL levels.

Medications along with diet and exercise enable increasing of the HDL level of the patient to above a selected minimum value, such as more than about 60 mg/dL. With the HDL level more than the LDL level (HDL>LDL), possibility of atherosclerotic plaque formation or progression approaches zero. This relative higher concentration of HDL than LDL maintains sufficient HDL including HDL2b to be efficient in picking up the LDL even prior to the LDL being deposited. Greatly reducing sugar and starch intake along with restricted saturated fat intake while consuming monounsaturated fat assists in attaining the HDL:LDL ratio greater than one.

Administering an HDL level increasing medication such as nicotinic acid raises the HDL level and also lowers both LDL levels and triglyceride levels. In addition to helping raise HDL levels including HDL2b, the nicotinic acid decreases levels of a lipoprotein (a) (Lp(a)), which is a particular LDL particle with an abnormal protein attached. Increased risks of rapid plaque buildup, heart disease and stroke correlate respectively with elevated levels of the Lp(a). Further, the nicotinic acid promotes change of LDL particle size from small to large.

Dropping the LDL level below the identified maximum value while increasing the HDL level above the selected minimum value can ensure that excess cholesterol in the adipose tissue and the plaques is mobilized and that there is a reverse transport of cholesterol from the adipose tissue and the plaques to the liver. Plaque regression can thereby return the arteries to normal conditions prior to atherosclerosis. Further, no additional plaque forms along the inner walls of the arteries as a result of both the LDL level, particularly small particle sizes of the LDL, not providing opportunity for deposition and the HDL>LDL causing pickup of the LDL by the HDL. In effect, the treatments described herein reverse heart disease.

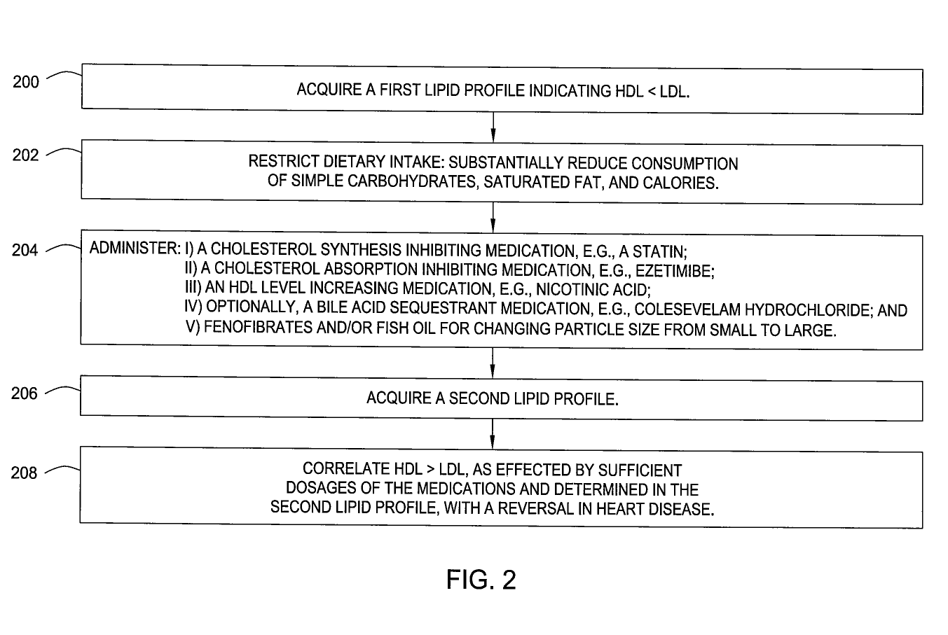

FIG. 2 shows an exemplary flow diagram according to an embodiment implementing aspects based on the foregoing. At an initial baseline step 200, treatment begins by acquiring a first lipid profile on a first blood sample of a patient. The first lipid profile indicates that the HDL level of the patient is less than the LDL level of the patient. Restricting dietary intake of the patient occurs at diet step 202. For example, dietary intake restrictions substantially reduce consumption of simple carbohydrates. Medication step 204 includes administering a combination of cholesterol altering medications to the patient such that the medications are effective to provide the HDL level greater than the LDL level. Next, acquiring a second lipid profile on a second blood sample of the patient occurs at follow-up monitoring step 206. At results detection step 208 based on the follow-up monitoring step 206, correlating the HDL level that is higher than the LDL level with the reversal in heart disease can include a physical identification within the lipid profile such as markings indicating target reached or color coding of the HDL and LDL levels.

For some embodiments, a method of reversing heart disease includes administering a combination of cholesterol altering medications to a patient having dietary intake restrictions such that the medications are effective to provide an HDL cholesterol level of the patient that is higher than an LDL cholesterol level of the patient. Further, a method, according to one embodiment, of detecting a reversal in heart disease includes acquiring a lipid profile on a blood sample of a patient, and correlating an HDL cholesterol level of the patient that is higher than an LDL cholesterol level of the patient with the reversal in heart disease. In still other embodiments, a method of treating heart disease includes administering a cholesterol synthesis inhibiting medication, a cholesterol absorption inhibiting medication, and an HDL level increasing medication to a patient, and selecting effective dosages of the medications sufficient to achieve, when combined with dietary intake restrictions, an HDL cholesterol level of the patient higher than an LDL cholesterol level of the patient.

### EXAMPLE 1

Fifty-six patients presented themselves for cardiac consultation during 2005 and 2006. Of the 56 patients, 26 (46.4%) were male and 30 (53.6%) were female. By race they were: African American (12.5%), Asian (17.9%), Caucasian (64.3%), and Hispanic (5.4%). Also, 46.4% indicated they exercise and 17.9% were smokers.

After measurement and consultation, the patients adhered to diet and drug regimens prescribed based on embodiments of the invention. Unlike prior approaches and drug treatment procedures where one might expect some subsequent coronary events among the patients without interventions such as angioplasty or coronary bypass surgery, none of the fifty-six patients after following the regimens prescribed experienced a post-treatment adverse coronary event. Table 1 shows results after a follow-up visit where the measurements were repeated.

TABLE 1

| | Average | Average |
|---|---|---|
| | Initial Visit | Follow-Up Visit |
| Total Cholesterol (mg/dl) | 210.21 | 131.09 |
| LDL (mg/dl) | 123.45 | 46.23 |
| HDL (mg/dl) | 58.79 | 67.89 |
| Triglycerides (mg/dl) | 139.71 | 84.88 |
| Percent LDL | 58.02% | 34.79% |
| Percent HDL | 28.08% | 52.04% |
| Percent Triglycerides | 13.20% | 13.17% |
| LDL/HDL ratio | 2.20 | .69 |
| Total Cholesterol/HDL ratio | 3.79 | 1.96 |
| Percent HDL2b (54 of 56 patients) | 20.69% | 26.78% |
| ApoB (mg/dl) (47 of 56 patients) | 100.11 | 54.09 |
| ApoA1 (mg/dl) (41 of 56 patients) | 169.37 | 165.95 |

Total cholesterol was lowered from 210.21 mg/dl to 131.09 mg/dl. LDL was lowered from 123.45 mg/dl to 46.23 mg/dl while HDL was increased from 58.79 mg/dl to 67.89 mg/dl. Although the percent of total cholesterol that is LDL was reduced from 58.02% to 34.79% and the percent that is HDL was increased from 28.08% to 52.04% (both statistically significant changes) the percent triglycerides remained at about 13% even though the amount of triglycerides was lowered from 139.71 mg/dl to 84.88 mg/dl.

### EXAMPLE 2

A 61 year old male presented himself for cardiac consultation on Feb. 20, 2006, when his blood was sent out for an advanced lipid profile. An electron beam computed tomography (EBCT) calcium scoring performed on Feb. 13, 2006 determined his calcium scoring was 2014. Prior to his initial visit on the 20^{th}, he took 10 mg daily of an atorvastatin as his pharmacologic lipid modification therapy, which was stopped. He then after his first consultation started taking a combination of cholesterol altering medications that included 10 mg of an ezetimibe and 40 mg of a simvastatin taken once a day, 1875 mg of a colesevelam taken two times daily, and 500 mg of niacin taken once daily.

The patient's initial blood work showed total cholesterol of 156, LDL of 87, HDL of 40 and triglycerides of 145. Further, his LDL particle size revealed almost 44% of it to be pattern B, small dense (goal set for less than about 15%), his HDL 2B was around 5% (goal set for more than about 35%), his ApoB was around 85 (goal set for less than about 60), and his Lpa was 31 (goal set for less than about 10). His Apo E genotype was 3/3, his LpPLA2 level was 170, his fibrinogen was 422, his insulin was 8, CRP was 0.7, his glucose was 97 and TSH was 1.0.

On Mar. 20, 2006 the patient returned for a follow-up visit and thereafter began taking a revised combination of cholesterol altering medications that included 10 mg of an ezetimibe and 20 mg of a simvastatin taken once a day, about 72.5 mg of a fenofibrate taken daily, 2000 mg of a fish oil twice a day, 500 mg of niacin taken once daily and increased to 1000 mg once a day in one month, and 1875 mg of a colesevelam taken twice a day. In addition to following drug regimens prescribed, the patient adhered to a low-calorie diet, exercise, and weight loss program as set forth herein. He lost close to 30 pounds in weight by Jun. 19, 2006, when he had a repeat limited blood work done that showed a total cholesterol of 115, LDL of 41, HDL of 63, and triglycerides of 54. Further, his LDL particle size changed from pattern B to pattern A, and his LDL 3A, 3B and 4B levels or percentage dropped from 44% to 24%.

On Oct. 20, 2006, the patient had another EBCT calcium scoring done which showed a calcium score of 2007. His calcium volume increased by about 3%, but his calcium score remained almost the same. The higher the calcium score on cardiac calcium testing, the more plaque is present in the arteries of the heart making the chance of having a heart attack higher. A calcium score of over 2000 and his age placed the patient in the 97th percentile (3% have higher scores) for calcium scoring. Expected natural progression of calcification predicted his calcium score in the course of these nine months to have gone up to about 2500 to 3000. However, nine months after treatment he had no progression. Based on these results, the patient halted the progression of atherosclerosis after modifying his diet, losing weight, and taking medications, which increased the HDL to 63 and dropped his LDL to 41 with change in particle size. Additionally, the drop in calcium score over the nine months indicated that progression halted in a few weeks upon commencing treatment and regression then ensued.

While the foregoing is directed to embodiments of the present invention, other and further embodiments of the invention may be devised without departing from the basic scope thereof, and the scope thereof is determined by the claims that follow.

1. A method of treating heart disease, comprising:
administering a combination of cholesterol altering medications to a patient having dietary intake restrictions such that the medications are effective in altering cholesterol to provide a high-density lipoprotein (HDL) cholesterol level of the patient that is higher than a low-density lipoprotein (LDL) cholesterol level of the patient.

2. The method of claim 1, wherein the medications comprise a statin, ezetimibe, colesevelam hydrochloride, and nicotinic acid.

3. The method of claim 1, wherein the medications comprise at least three different compounds with each of the compounds representing a different type of category selected from cholesterol synthesis inhibitors, cholesterol absorption inhibitors, HDL level increasers, and bile acid sequestrants.

4. The method of claim 1, wherein the medications comprise at least three medications selected from a statin, ezetimibe, colesevelam hydrochloride and nicotinic acid.

5. The method of claim 1, wherein the HDL cholesterol level is greater than 60 milligrams per deciliter (mg/dL).

6. The method of claim 1, wherein the LDL cholesterol level is less than 50 milligrams per deciliter (mg/dL).

7. The method of claim 1, wherein the HDL cholesterol level is greater than 60 milligrams per deciliter (mg/dL) and the LDL cholesterol level is less than 50 mg/dL.

8. The method of treating heart disease, comprising:
administering a combination of cholesterol altering medications to a patient having dietary intake restrictions such that the medications are effective in altering cholesterol to provide a high-density lipoprotein (HDL) cholesterol level of the patient that is higher than a low-density lipoprotein (LDL) cholesterol level of the patient, wherein the dietary intake restrictions substantially exclude intake or simple carbohydrates.

9. The method of claim 1, wherein approximately 50% of solids consumed according to the dietary intake restrictions are complex carbohydrates and approximately 50% of the solids are selected from at least one of tofu, substantially soy products, brown rice, wild rice, at least substantially fat free meats, egg whites, nuts, avocados, olives and no sugar yogurt.

10. A method of detecting a heart disease, treatment criteria, comprising:
acquiring a lipid profile on a blood sample of a patient; and
assessing the lipid profile by establishing that a high-density lipoprotein (HDL) cholesterol level of the patient that is higher than a low-density lipoprotein (LDL) cholesterol level of the patient corresponds with plaque regression and thereby identifies the criteria.

11. The method of claim 10, wherein the assessing includes a physical identification within the lipid profile.

12. The method of claim 11, further comprising, before acquiring the lipid profile, acquiring an initial lipid profile of the patient prior to a treatment to reverse heart disease, wherein the initial lipid profile indicates that the HDL cholesterol level is lower than the LDL cholesterol level.

13. A method of treating heart disease, comprising:
administering a cholesterol synthesis inhibiting medication to a patient;
administering a cholesterol absorption inhibiting medication to the patient;
administering a high-density lipoprotein (HDL) level increasing medication to the patient; and
selecting effective dosages of the medications sufficient to achieve, when combined with dietary intake restrictions, an HDL cholesterol level of the patient higher than a low-density lipoprotein (LDL) cholesterol level of the patient.

14. The method of claim 13, further comprising administering a bile acid sequestrant medication to the patient.

15. The method of claim 13, wherein the cholesterol synthesis inhibiting medication comprises a statin, the cholesterol absorption inhibiting medication comprises ezetimibe, and the HDL level increasing medication comprises nicotinic acid.

16. The method of claim 15, further comprising administering a bile acid sequestrant medication to the patient.

17. The method of claim 13, wherein the HDL cholesterol level is greater than 60 milligrams per deciliter (mg/dL).

18. The method of claim 13, wherein the LDL cholesterol level is less than 50 milligrams per deciliter (mg/dL).

19. The method of claim 13, wherein the HDL cholesterol level is greater than 60 milligrams per deciliter (mg/dL) and the LDL cholesterol level is less than 50 mg/dL.

20. A method of treating heart disease, comprising:
administering a cholesterol synthesis inhibiting medication to a patient;
administering a cholesterol absorption inhibiting medication to the patient;
administering a high-density lipoprotein (HDL) level increasing medication to the patient; and
selecting effective dosages of the medications sufficient to achieve, when combined with dietary intake restrictions, an HDL cholesterol level of the patient higher than a low-density lipoprotein (LDL) cholesterol level of the patient, wherein the dietary intake restrictions substantially exclude intake of simple carbohydrates.
